(19) United States
(12) Patent Application Publication
(10) Pub. No.: US 2006/0159644 A1
(43) Pub. Date: Jul. 20, 2006

(54) Compositions comprising a block copolymer, an amino silicone and a nonpolymeric thickener, and uses thereof

(76) Inventors: Lydia Panangatte, Poissy (FR); Franck Giroud, Clichy (FR); Claude Dubief, le Chesnay (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/315,140

(22) Filed: Dec. 23, 2005

Related U.S. Application Data
(60) Provisional application No. 60/642,994, filed on Jan. 12, 2005.

(30) Foreign Application Priority Data
Dec. 23, 2004 (FR) 04 13800

(51) Int. Cl. A61K 8/81

(52) U.S. Cl. 424/70.11; 424/70.16

(57) ABSTRACT

The disclosure provides cosmetic compositions comprising, in a cosmetically acceptable medium, at least one block copolymer comprising at least a first hydrophilic block and at least a second block that is chosen from a second hydrophilic block, wherein the second hydrophilic block is different than the first hydrophilic block, and a hydrophobic block, wherein the hydrophilic block(s) represent at least 30% by weight of the at least one block copolymer, at least one amino silicone, and at least one nonpolymeric thickener chosen from at least one fatty alcohol and at least one amide. The present disclosure also provides uses of the compositions for the disentangling, smoothing, and imparting sheen to keratin fibers such as hair.

This application claims the benefit of U.S. Provisional Application No. 60/642,994, filed Jan. 12, 2005, the contents of which are incorporated herein by reference. This application also claims the benefit of priority under 35 U.S.C. § 119 to French Patent Application No. 04 13800, filed Dec. 23, 2004, the contents of which are also incorporated herein by reference.

The present disclosure provides cosmetic compositions comprising, in a cosmetically acceptable medium, at least one block copolymer comprising at least a first hydrophilic block and at least a second block that is chosen from a second hydrophilic block, wherein the second hydrophilic block is different than the first hydrophilic block, and a hydrophobic block, wherein the hydrophilic block(s) represent at least 30% by weight of the at least one block copolymer, at least one amino silicone, and at least one nonpolymeric thickener chosen from at least one fatty alcohol and at least one amide.

The disclosure also provides uses of those compositions and methods employing those compositions for disentangling and smoothing out of keratin fibers and for giving keratin fibers sheen.

It is well known that hair that has been sensitized, i.e., damaged and/or made brittle, to varying degrees by atmospheric agents, by mechanical treatments, or chemical treatments, such as dyeing, bleaching and/or permanent-waving, are often difficult to disentangle and to style, and lack softness.

In order to attempt to solve this problem, those skilled in the art already use, in care compositions for keratin materials such as hair, conditioners, in particular cationic surfactants, cationic polymers, and silicones.

However, while these compositions make it possible, to a certain extent, to improve the disentangling and the softness of the hair, they are unfortunately also accompanied by certain cosmetic effects judged to be undesirable on hair, namely making the hair feel lank (i.e., lacking lightness) and not smoothed out (hair not homogeneous from the root to the tip), and provide insufficient sheen.

In addition, the use of cationic polymers for this purpose has various drawbacks. Because of their strong affinity for the hair, some of these polymers become substantially deposited during repeated use, and lead to undesirable effects such as an unpleasant laden feel, stiffening of the hair, and adhesion between the fibers, which affects the styling. These drawbacks are accentuated in the case of fine hair, which lacks liveliness and volume.

The current cosmetic compositions containing conditioners do not therefore provide complete satisfaction.

In addition, the formulation of the current cosmetic compositions is based on the use of cationic surfactants, which are poorly tolerated by aquatic fauna and flora.

After numerous studies, the Applicants have determined that the above problems can be solved by applying to hair a composition comprising, in a cosmetically acceptable medium, at least one block copolymer comprising at least a first hydrophilic block and at least a second block that is chosen from a second hydrophilic block, wherein the second hydrophilic block is different than the first hydrophilic block, and a hydrophobic block, wherein the hydrophilic block(s) represent at least 30% by weight of the at least one block copolymer, at least one amino silicone, and at least one nonpolymeric thickener chosen from at least one fatty alcohol and at least one amide, with the proviso that at least one block copolymer is not chosen from a block copolymer of ethylene oxide and propylene oxide, a block copolymer comprising urethane units, and a block copolymer comprising siloxane units.

The Applicants have demonstrated that the use of certain of block copolymers, described in greater detail hereinafter, in combination with an amino silicone and a nonpolymeric thickener chosen from fatty alcohols and amides makes it possible to overcome the drawbacks of the compositions of the prior art.

The compositions according to the present application thus make it possible to improve disentangling of the hair while at the same time giving the hairstyle good cosmetic properties such as lightness, smoothness and sheen, which are maintained after multiple applications. They also make it possible to avoid the drawbacks generally encountered when using cationic polymers.

Consequently, provided are cosmetic compositions comprising, in a cosmetically acceptable medium:
at least one block copolymer comprising at least a first hydrophilic block and at least a second block that is chosen from a second hydrophilic block, wherein the second hydrophilic block is different than the first hydrophilic block, and a hydrophobic block, wherein the hydrophilic block(s) represent at least 30% by weight of the at least one block copolymer,
at least one amino silicone, and
at least one nonpolymeric thickener chosen from at least one fatty alcohol and at least one amide.

In some embodiments, the at least one block copolymer is not chosen from a block copolymer of ethylene oxide and propylene oxide, a block copolymer comprising urethane units, and a block copolymer comprising siloxane units.

The present application discloses the use of the compositions, for the disentangling of, the smoothing and giving sheen to keratin fibers such as hair, and the maintaining of these properties after multiple applications.

Other characteristics, aspects, subjects and advantages of the present disclosure will become even more clearly apparent on reading the description and the examples that follow.

In some embodiments, the cosmetic compositions are nondetergent cosmetic compositions. As used herein, the term “nondetergent” means that the composition does not contain more than 4% by weight of surfactants relative to the total weight of the composition.

As used herein, the term “block copolymer” denotes the copolymer used in the compositions described herein, i.e., a block copolymer comprising at least a first hydrophilic block and at least a second block that is chosen from a hydrophilic block other than the first hydrophilic block and a hydrophobic block, wherein the hydrophilic block(s) represent at least 30% by weight of the block copolymer.

The block copolymers that can be used include linear and star, diblock, triblock, and multiblock copolymers that, in water, may react in a manner chosen from solubilizing spontaneously, dispersing spontaneously, solubilizing with neutralization, and dispersing with neutralization. In some embodiments, the at least one block copolymer is a star copolymer and is chosen from diblock copolymers, triblock copolymers, and multiblock copolymers. In other embodiments, the at least one block copolymer is a linear copolymer and is chosen from diblock copolymers, triblock copolymers, and multiblock copolymers.

The block copolymers that can be used may include hydrophilic and water-soluble copolymers comprising at least two different hydrophilic blocks, as well as amphiphilic copolymers comprising at least one hydrophobic block and at least one hydrophilic block. These copolymers may be chosen from anionic copolymers, nonionic copolymers, and amphoteric copolymers, for example, anionic copolymers.

As used herein, the term “water-soluble or water-dispersible monomer or polymer” includes monomers and polymers which, at the concentration of 0.1% with respect to active material in water at 25° C., give, spontaneously or after neutralization with an acid or a base, a macroscopically homogeneous and transparent solution, i.e., a solution having a transmittance value at a wavelength of 500 nm through a sample 1 cm thick of at least 70%, for example, at least 80%. Monomers and polymers that do not meet this criterion are said to be hydrophobic.

The ability of amphiphilic block copolymers to be dissolved and even finely dispersed in water is linked to the proportion of hydrophilic blocks. This proportion is at least 30% by weight, for example, greater than or equal to 60% by weight, but does not exceed 97% by weight.

As used herein, the term “hydrophilic block” is intended to mean a block comprising at least 75 mol % of monomers chosen from water-soluble monomers, water-solubilizable monomers, and monomers that are water-dispersible with neutralization. The hydrophilic block may therefore comprise up to 25 mol % of at least one hydrophobic monomer as defined above. This proportion may be up to 10 mol %, for example, up to 5 mol %.

As used herein, the term “hydrophobic block” means a block polymer comprising at least 75 mol % of hydrophobic monomers. The hydrophobic block may therefore comprise up to 25 mol % of at least one water-soluble or water-dispersible monomer as defined above. This proportion may be less than or equal to 10 mol %, for example, less than or equal to 5 mol %.

Block copolymers that may be used also include those in which the hydrophilic blocks and the hydrophobic blocks comprise water-soluble or water-dispersible monomers, and hydrophobic monomers, respectively.

These hydrophilic blocks and the hydrophobic blocks may be chosen from homopolymeric blocks and copolymeric blocks comprising two or more different monomers of the same type.

The number-average molecular mass of each block, whether it is chosen from hydrophilic blocks and hydrophobic blocks, and copolymeric blocks and homopolymeric blocks, may range from 500 to 1,000,000, for example, from 500 to 500,000. The polydispersity index (Mw/Mn) of the copolymer may range from 1.01 to 3.00, for example, from 1.10 to 2.50.

The monomers chosen from water-soluble monomers and water-dispersible monomers forming the hydrophilic blocks of the block copolymers may be chosen from anionic monomers, nonionic monomers, and cationic monomers, and may be used alone or in the form of a mixture comprising two or more different monomers.

Examples of anionic water-soluble and water-dispersible monomers include ethylenically unsaturated carboxylic acids, such as acrylic acid, methacrylic acid, itaconic acid, fumaric acid, crotonic acid, maleic acid, 2-acrylamido-2-methylpropanesulphonic acid, styrenesulphonic acid, vinylsulphonic acid, and vinylphosphonic acid.

Nonionic water-soluble and water-dispersible monomers include, inter alia, acrylamide, C₁₋₆ N-alkyl acrylamides, C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol (copolymerized in the form of vinyl acetate and then hydrolyzed), ethylene oxide, hydroxyethyl acrylate, hydroxypropyl acrylate, hydroxyethyl methacrylate, and hydroxypropyl methacrylate.

Cationic water-soluble and water-dispersible monomers may include, for example, dimethyidiallylammonium chloride, methylvinylimidazolium chloride, 2-vinyl-pyridine, 4-vinylpyridine, 2-methyl-5-vinylpyridine, vinylamine, and monomers of formula
H₂C═CR₁—CO—X₂
wherein:

R₁ is chosen from hydrogen and methyl,

X₂ is chosen from

a C₁₋₆ linear hydrocarbon-based group bearing at least one substituent chosen from primary amino, secondary amino, tertiary amino, and quaternary amino;

a C₁₋₆ branched hydrocarbon-based group bearing at least one substituent chosen from primary amino, secondary amino, tertiary amino, and quaternary amino;

a group of formula NHR₂; and

a group of formula NR₂R₃;

wherein R₂ and R₃ each are, independently of one another, chosen from C₁₋₆ linear hydrocarbon-based groups bearing at least one substituent chosen from primary amino, secondary amino, tertiary amino, and quaternary amino; and C₁₋₆ branched hydrocarbon-based groups bearing at least one substituent chosen from primary amino, secondary amino, tertiary amino, and quaternary amino.

In certain embodiments, the hydrophobic monomers forming the hydrophobic blocks of the block copolymers are chosen from

vinylaromatic monomers such as styrene;

alkylated derivatives of vinylaromatic monomers for example, 4-butylstyrene, alpha-methylstyrene and vinyltoluene;

dienes such as butadiene and 1,3-hexadiene;

alkylated derivatives of dienes, such as isoprene and dimethylbutadiene;

chloroprene;

acrylates chosen from C₁₋₁₀ alkylacrylates, C₆₋₁₀ arylacrylates, and C₆₋₁₀ aralkyl acrylates;

methacrylates chosen from C₁₋₁₀ alkylmethacryaltes, C₆₋₁₀ arylmethacrylates, and C₆₋₁₀ aralkyl methacrylates, for example, methyl, ethyl, n-butyl, 2-ethylhexyl, tert-butyl, isobornyl, phenyl, and benzyl (meth)acrylate;

vinyl acetate;

vinyl ethers of formula CH₂═CH—O—R;

allyl ethers of formula CH₂═CH—CH₂—O—R wherein R is C₁₋₆ alkyl;

acrylonitrile;

vinyl chloride;

vinylidene chloride;

caprolactone;

ethylene;

propylene;

fluorinated vinyl monomers

vinyl monomers comprising a perfluoro chain, such as fluoroalkyl acrylates and methacrylates; and

alkyl alpha-fluoroacrylates.

In certain embodiments, the linear block copolymers are chosen from:

diblock copolymers of the formula AB,

triblock copolymers of the formula ABA,

triblock copolymers of the formula BAB, and

multiblock copolymers comprising at least two hydrophilic blocks and at least two hydrophobic blocks arranged alternately,

wherein each A represents a hydrophilic block and each B represents a hydrophobic block, it being possible for the A blocks of the same polymer to be identical or different and it being possible for the B blocks of the same polymer to be identical or different.

Diblock copolymers and triblock copolymers comprising a hydrophilic central block and two hydrophobic side blocks may be used.

The block polymers can be prepared by synthetic processes conventionally used to obtain block polymers. Examples of anionic and cationic polymerizations, and controlled radical polymerization are provided in “New Method of Polymer Synthesis,” Blackie Academic & Professional, London, 1995, Volume 2, page 1, and Trends Polym. Sci. 4, page 183 (1996) by C. J. Hawker, which may be carried out according to various processes, for example, atom transfer radical polymerization (or ATRP) (JACS, 117, page 5614 (1995), by Matyjasezwski et al.), a method employing radicals such as nitroxides (Georges et al., Macromolecules, 1993, 26, 2987) or radical addition-fragmentation chain transfer such as the MADIX (Macromolecular Design via the Interchange of Xanthate) process (Charmot D., Corpart P., Adam H., Zard S. Z., Biadatti T., Bouhadir G., Macromol. Symp., 2000, 150, 23).

These processes may also be used to obtain just one of the two types of blocks of the polymers, the other block being introduced into the final polymer via the initiator used or by means of a coupling reaction between the hydrophilic and hydrophobic blocks.

Star polymers and dendrimers are highly branched polymers and oligomers that have a well-defined chemical structure; they are said to be “perfect” hyperbranched polymers. Dendrimers comprise a core, a determined number of generations of spindles, (or branches), and terminal groups. The generations of spindles comprise structural units which may be identical for the same generation of spindles and which may be identical or different for different generations of spindles. The generations of spindles extend radially in a geometrical progression from the core. The terminal groups of a dendrimer of the Nth generation are the terminal functional groups of the spindles of the Nth generation or terminal generation. Such polymers are described in D. A. Tomalia, A. M. Naylor and W. A. Goddard III, Angewandte Chemie, Int. Ed. Engl. 29, 138-175 (1990); C. J. Hawker and J. M. J. Frechet, J. Am. Chem Soc., 112, 7638 (1990); B I Volt, Acta Polymer., 46, 87-99 (1995); N. Ardoin and D. Astruc. Bull. Soc. Chim. Fr 132, 875-909 (1995); G. R. Newkome, C. N. Moorefield, F. Vögtle, Dendritic Molecules, VCH Verlagegesellschaft, 1996.

The amount of block copolymers in the compositions depends on many parameters, including the molecular mass of the block copolymers, the number and the size of the hydrophilic and, optionally, hydrophobic blocks, and optionally the viscosity of the desired compositions.

For example, a copolymers chosen from:
poly(styrene-b-acrylic acid);
poly(methylacrylamide-b-acrylic acid);
poly(quaternized trimethylammonium ethyl acrylate-b-acrylamide) diblock copolymer; and
poly(styrene-b-acrylic acid-b-styrene) triblock copolymer,
   may be used.

The block copolymers may be present in the compositions in an amount ranging from 0.01 to 20% by weight of active material, for example, from 0.1 to 5% by weight of active material, relative to the total weight of the composition.

The compositions may comprise at least one amino silicone. As used herein, the term “amino silicone” denotes any silicone comprising at least one group chosen from primary amino groups, secondary amino groups, tertiary amino groups, and quaternary ammonium groups. As used herein, the terms “amino silicone” and “amino functional silicone” are used interchangeably to denote the amino silicone used in the compositions described herein.

Amino silicones include, for example:
(a) polysiloxanes of formula (VIII): 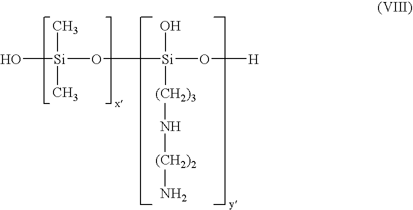
wherein x′ and y′ are each integers that may vary depending on the molecular weight, generally chosen such that the weight-average molecular weight ranges from 5000 to 500,000;
(b) amino silicones of formula (III):
   R′ₐG₍₃₋ₐ₎-Si(OSiG₂)ₙ—(OSiG_{b}R′_{(2-b)})ₘ—O—SiG₍₃₋ₐ₎-R′ₐ (III)
wherein:
   each G, which may be identical or different, is chosen from hydrogen, phenyl, OH, C₁-C₈ alkyl (for example methyl), and C₁-C₈ alkoxy, (for example methoxy),
   each a, which may be identical or different, denotes the number 0 or is an integer chosen from 1, 2, and 3, for example 0,
   b is chosen from 0 and 1, for example 1,
   m and n are each numbers chosen such that the sum (n+m) may range from 1 to 2000, for example, the sum (n+m) may range from 50 to 150,
   each R′, which may be identical or different, is a monovalent radical of the formula —C_{q}H_{2q}L wherein q is a number chosen from 2, 3, 4, 5, 6, 7, and 8 and L is an optionally quaternized amino group chosen from:
—NR″—Q-N(R″)₂,
—N(R″)₂,
—N⁺(R″)₃A⁻,
—N⁺H(R″)₂A⁻,
—N⁺H₂(R″)A⁻,
—N(R″)-Q-N⁺R″H₂A⁻,
—NR″-Q-N⁺(R″)₂H A⁻, and
—NR″-Q-N⁺(R″)₃ A⁻,
   wherein R″ is chosen from hydrogen, phenyl, benzyl, and a monovalent saturated hydrocarbon-based radical, for example, an alkyl radical having from 1 to 20 carbon atoms; Q is chosen from a linear group of formula Cᵣ_{H}₂ᵣ and a branched group of formula CᵣH₂ᵣ, wherein r is an integer chosen from 2, 3, 4, 5, and 6, for example an integer chosen 2, 3, and 4; and A⁻ is a halide ion such as, for example, fluoride, chloride, bromide, and iodide.

In some embodiments, n is a number ranging from 0 to 1999, for example, ranging from 49 to 149. In some embodiments, m is a number ranging from 1 to 2000, for example, ranging from 1 to 10.

Such amino silicones include silicones called “trimethylsilylamodimethicone,” corresponding to formula (X): 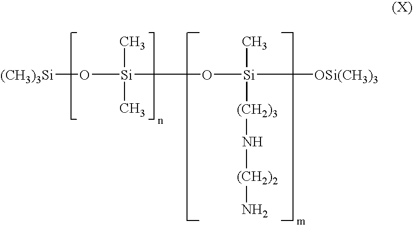
wherein n and m are as described above (cf. formula III).

Such polymers are described, for example, in patent application EP-A-95238.

Another group of amino silicones that may be used include silicones of formula (I) and (II). Amino silicones of formula (I) have the structure: 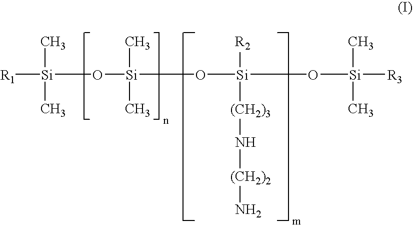
wherein:

m and n are integers chosen such that the sum (n+m) may range from 1 to 1000, for example, the sum (n+m) may range from 50 to 250 or from 100 to 200, wherein

n is a number ranging from 0 to 999, for example, from 49 to 249 or from 125 to 175,

m is a number ranging from 1 to 1000, for example, from 1 to 10 or from 1 to 5,

R₁, R₂, and R₃, which may be identical or different, are independently chosen from hydroxyl and C₁-C₄ alkoxy, wherein at least one of the radicals R₁, R₂, and R₃ is alkoxy, such as methoxy.

The hydroxyl/alkoxy molar ratio may range from 0.2:1 to 0.4:1, for example, from 0.25:1 to 0.35:1 or may be equal to about 0.3:1.

The weight-average molecular mass of the silicone may range from 2000 to 1,000,000, for example, from 3500 to 200,000.

Amino silicones of formula (II) have the structure: 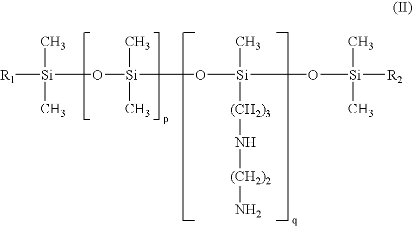
wherein:

p and q are numbers chosen such that the sum (p+q) may range from 1 to 1000, for example, the sum (p+q) may range from 50 to 350, and in some embodiments, the sum (p+q) may range 150 to 250, wherein

p is a number ranging from 0 to 999, for example, from 49 to 349 or from 159 to 239, and

q is a number ranging from 1 to 1000, for example, from 1 to 10 or from 1 to 5;

R₁ and R₂, which are different, are independently chosen from hydroxyl and C₁-C₄ alkoxy, wherein at least one of the radicals R₁ or R₂ is alkoxy, such as methoxy.

The hydroxyl/alkoxy molar ratio may range from 1:0.8 to 1:1.1, for example, from 1:0.9 to 1:1, or may be equal to about 1:0.95.

The weight-average molecular mass of the silicone may range from 2000 to 200,000, for example, from 5000 to 100,000 or from 10,000 to 50,000.

The weight-average molecular masses of these amino silicones may be measured by gel permeation chromatography (GPC) at ambient temperature in polystyrene equivalent. For example, the eluent may be THF at a flow rate of 1 ml/min using μ styragel columns, injecting 200 μl of a 0.5% by weight solution of silicone in THF, and detected by refractometry and measured with a UV-meter.

Commercial products corresponding to these silicones of formulas (I) and (II) may further comprise one or more other amino silicones whose structure is different from formulas (I) and (II).

A product containing amino silicones of structure (I) is sold by the company Wacker under the name BELSIL® ADM 652.

A product containing amino silicones of structure (II) is sold by Wacker under the name Fluid WR 1300®.

In some embodiments, these amino silicones are used in the form of an oil-in-water emulsion. The oil-in-water emulsion may also comprise one or more surfactants.

The surfactants may be of any nature, for example, cationic surfactants and nonionic surfactants may be used.

The number-average size of the silicone particles in the emulsion is may range from 3 to 500 nm.

In some embodiments, amino silicones of formula (II), microemulsions for which the average size of the particles ranges from 5 to 60 nm or even from 10 to 50 nm, are used.

Thus, it is possible to use microemulsions of amino silicone of formula (II) provided under the names Finish CT 96 E® and SLM 28020® by the company Wacker.

In some embodiments, the amino silicone is chosen such that the contact angle with the water of a hair treated with a composition containing 2% (active material) of the amino silicone ranges from 90 to 180°, for example, from 90 to 130°.

To measure the contact angle, the amino silicone may be solubilized or dispersed in a solvent for the amino silicone or for the amino silicone emulsion (for example, hexamethyldisiloxane or water depending on the hydrophilicity of the silicone).

The composition comprising an amino silicone of formulas (I) and/or (II) may be such that the contact angle with the water of a hair treated with the composition ranges from 90 to 180° or even from 90 to 130°.

The contact angle measurement is based on immersing a hair in distilled water. The force exerted by the water on the hair during its immersion in distilled water and during its removal is determined. The force thus measured is directly linked to the contact angle θ between the water and the surface of the hair. The hair is said to be hydrophilic when the angle θ ranges from 0 to 90° and hydrophobic when the angle ranges from 90 to 180°.

The test is carried out with locks of natural hair that have been bleached under the same conditions and then washed.

Each 1 g lock is placed in a crystallizing dish 75 mm in diameter and then covered uniformly with 5 ml of the composition to be tested. The lock is left for 15 minutes at ambient temperature and then rinsed with distilled water for 30 seconds. The drained lock is left in the open air until it is completely dry.

For each evaluation, 10 locks that have undergone the same treatment are analyzed. Each sample, attached to a precision microbalance, is immersed via the tip in a container filled with distilled water. The DCA balance (“dynamic contact angle analyzer”), from the company Cahn Instruments, allows the force (F) exerted by the water on the hair to be measured.

In parallel, the perimeter (P) of the hair is measured by means of observation under a microscope.

The contact angle of the hair on the water may be determined using mean wetability force on ten hairs and the cross section of the analyzed hairs according to the formula:
F=P*ΓIv*cos θ
where F is the wetability force in newtons, P is the perimeter of the hair in meters, ΓIv is the liquid/water vapor interface tension in J/m², and θ is the contact angle.

The product SLM 28020 from Wacker at 12% in water (i.e., 2% of amino silicone) gives a contact angle of 93° according to the test described above.

The product BELSIL ADM 652 from Wacker at 2% in hexamethyldisiloxane (i.e., 2% of amino silicone) gives a contact angle of 111° according to the test described above.

Another group of amino silicones corresponding to this definition is represented by formula (VI) below: 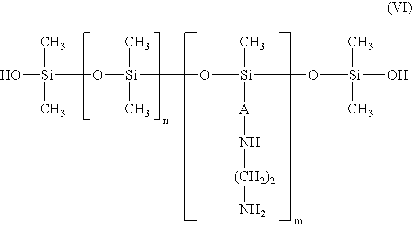
wherein:
m and n are numbers chosen such that the sum (n+m) ranges from 1 to 2000, for example, from 50 to 150, wherein:
n is a number ranging from 0 to 1999, for example, from 49 to 149, and
m is a number ranging from 1 to 2000, for example, from 1 to 10;
A is a chosen from linear alkylene radicals and branched alkylene radicals, each having from 4 to 8 carbon atoms, for example, chosen from 4, 5, 6, 7, and 8 carbon atoms. In some embodiments, A is linear.

The weight-average molecular mass of the amino silicones may range from 2000 to 1,000,000, for example, from 3500 to 200,000.

The weight-average molecular masses of these amino silicones may be measured by gel permeation chromatography (GPC) at ambient temperature as polystyrene equivalent using p styragel columns and an eluent of THF with a flow rate of 1 ml/min, injecting 200 μl of a 0.5% by weight solution of silicone in THF, and detecting by refractometry and measured with a UV-meter.

The viscosity at 25° C. of the amino silicone may be greater than 25,000 cSt (mm²/s), for example, ranging from 30,000 to 200,000 cSt (mm²/s) or from 30,000 to 150,000 cSt (mm²/s).

These amino silicones may have an amine number of less than or equal to 0.4 meq/g, for example, ranging from 0.001 to 0.2 meq/g or ranging from 0.01 to 0.1 meq/g.

The amine number is the number of amine milliequivalents per gram (meq/g) of compound. This number is determined in a conventional manner using titration methods using colored indicator or by potentiometric titration.

In some embodiments, the amino silicones are used in the form of oil-in-water emulsions. The oil-in-water emulsions may further comprise one or more surfactants.

The surfactants may be of any nature, for example, cationic surfactants and/or nonionic surfactants may be used.

The number-average size of the silicone particles in the emulsion may range from 3 to 500 nm, for example, from 5 to 300 nm, from 10 to 275 nm, or even from 150 to 275 nm.

A silicone corresponding to this formula is, for example, DC2-8299 from Dow Corning.

Amino silicones corresponding to formula (XI) may also be used: 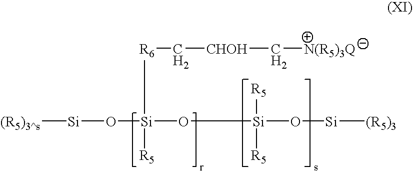
wherein:
R₅ is a monovalent hydrocarbon-based radical having from 1 to 18 carbon atoms, for example, chosen from C₁-C₁₈ alkyl and C₂-C₁₈ alkenyl, such as methyl;
R₆ is a divalent hydrocarbon-based radical, for example, chosen from a C₁-C₁₈ alkylene radical and a C₁-C₁₈ divalent alkyleneoxy radical, such as a C₁-C₈ radical connected to the Si via a silicon-carbon (SiC) bond;
Q⁻ is an anion chosen from a halide ion, e.g., chloride, and carboxylate ion, e.g., acetate; r is a mean statistical value, ranging from 2 to 20, for example, 2, 3, 4, 5, 6, 7, and 8; and
s is a mean statistical value, ranging from 20 to 200, for example, ranging from 20 to 50.

Such amino silicones are described U.S. Pat. No. 4,185,087.

Other silicones that may be used include quaternary ammonium silicones of formula (XII): 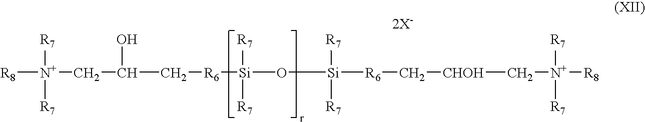
wherein:
each R₇, which may be identical or different, is a monovalent hydrocarbon-based radical having from 1 to 18 carbon atoms, for example, chosen from C₁-C₁₈ alkyl, C₂-C₁₈ alkenyl, and a ring comprising 5 or 6 carbon atoms, for example methyl;
each R₆, which may be identical or different, is a divalent hydrocarbon-based radical, for example, chosen from C₁-C₁₈ alkylene and C₁-C₁₈ divalent alkyleneoxy, for example C₁-C₈, connected to the Si via an SiC bond;
each R₈, which may be identical or different, is chosen from hydrogen and a monovalent hydrocarbon-based radical having from 1 to 18 carbon atoms, such as C₁-C₁₈ alkyl, C₂-C₁₈ alkenyl, and -R₆—NHCOR₇;
X⁻ is an anion, for example, chosen from halide ions and carboxylate ions, e.g., chloride, and acetate; and
r is a mean statistical value ranging from 2 to 200, for example, ranging from 5 to 100.

These silicones are, for example, described in application EP-A-0530974.

Other amino silicones that may be used include amino silicones of formula (XIII): 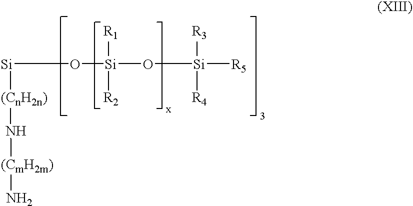
wherein:
R₁, R₂, R₃ and R₄, which may be identical or different, are chosen from C₁-C₄ alkyl and phenyl,
R₅ is chosen from C₁-C₄ and hydroxyl,
n is an integer ranging from 1 to 5, for example, 1, 2, 3, 4, and 5,
m is an integer ranging from 1 to 5, for example, 1, 2, 3, 4, and 5, and
x is chosen such that the amine number ranges from 0.01 to 1 meq/g.

Polyoxyalkylenated amino silicones of the formula (XY)ᵢ may also be used, wherein X is a polysiloxane block and Y is a polyoxyalkylenated block comprising at least one amine group that may comprise repeat units of the general formula below:
[SiMe₂—O—(SiMe₂O)ₓSiMe₂—R—N(H)—R′—O—(C₂H₄O)ₐ—(C₃H₆O)_{b}—R′—N(H)—R-] (IV)
wherein:
a is an integer greater than or equal to 1, for example, ranging from 5 to 200 or from 5 to 100;
b is an integer ranging from 0 to 200, for example, from 4 to 200 or from 5 to 100;
x is an integer ranging from 1 to 10,000, for example, from 10 to 5000;
each R, which may be identical or different, is a divalent organic group linked to the adjacent silicon atom via a carbon-silicon bond and to a nitrogen atom, and

each R′, which may be identical or different, is a divalent organic group linked to the adjacent oxygen atom via a carbon-oxygen bond and to a nitrogen atom.

In some embodiments, R is a C₂-C₁₂ hydrocarbon-based radical optionally comprising one or more heteroatoms such as oxygen. In certain embodiments, R is chosen from ethylene, linear propylene, branched propylene, linear butylene, branched butylene, and —CH₂CH₂CH₂OCH(OH)CH₂—.

In some embodiments, R′ is a C₂-C₁₂ hydrocarbon-based radical optionally comprising one or more heteroatoms such as oxygen. For example, R′ may be chosen from a divalent alkylene radical, such as ethylene, linear propylene, branched propylene, linear, butylene, and branched butylene.

In some embodiments, the siloxane blocks represent from 50 to 95 mol % relative to the total weight of the silicone, e.g., from 70 to 85 mol %.

In some embodiments, the amine content ranges from 0.02 to 0.5 meq/g of copolymer in a 30% solution in dipropylene glycol, for example, from 0.05 to 0.2 meq/g.

In some embodiments, the weight-average molecular weight of the silicone of formula (IV) ranges from 5000 to 1,000,000, for example, from 10,000 to 200,000.

One silicone of formula (IV) is sold under the trademark Silsoft A-843 Organosilicone Copolymer by OSI.

In some embodiments, the silicones that are used the polysiloxanes comprising amino groups of the families of formulae (I), (II), (III), (VI), (VIII), and (X).

The amino silicones may be used in an amount ranging from 0.01 to 20% by weight relative to the total weight of the composition. In some embodiments, the amount ranges from 0.1 to 15% or even from 0.5 to 10% by weight, relative to the total weight of the composition.

The nonpolymeric thickeners may be chosen from chosen from fatty alcohols and amides.

As used herein, the term “fatty alcohol” means an alcohol comprising a chain chosen from C₁₀-C₃₀ linear alkyl chains, C₁₀-C₃₀ branched alkyl chains, C₁₀-C₃₀ linear alkylene chain, and C₁₀-C₃₀ branched alkylene chains. The fatty alcohols that may be used include stearyl alcohol, myristic alcohol, cetyl alcohol, and mixtures thereof. In some embodiments, mixtures of stearyl alcohol and of cetyl alcohol are used.

Amides that may be used in the compositions include amides comprising a C₁₂-C₂₂, e.g., C₁₆-C₁₈, fatty chain. These amides may also be alkanolamides.

Nonpolymeric thickeners may be used in an amount ranging from 0.5 to 10%, e.g., ranging from 2 to 6% by weight, relative to the total weight of the composition.

The cosmetically acceptable medium may consist only of water, or be a mixture of water and of one or more cosmetically acceptable water-miscible solvents, such as C₁-C₄ lower alcohols, such as ethanol, isopropanol, tert-butanol and n-butanol, and polyols such as propylene glycol and glycerol.

The pH of the compositions generally ranges from 3 to 9, such as from 4 to 7.

The compositions may also comprise cosmetic additives and/or formulation adjuvants such as anionic, cationic, amphoteric surfactants; nonionic surfactants; additional anionic polymers, cationic polymers, amphoteric polymers, and nonionic polymers other than the block copolymers described herein; pearlescent agents; opacifiers; pigments and dyes; fragrances; plant, animal oils; synthetic oils; additional silicones and organosilicones other than the amino silicones described herein; silicone gums and resins; waxes, including ceramides, organic microparticles, organic nanoparticles, mineral microparticles, and mineral nanoparticles; organic UV-screening agents; mineral UV-screening agents; free-radical scavengers; vitamins; peptides; proteins; amino acids, such as basic amino acids; antidandruff agents; antifungal agents; agents for activating hair regrowth; plasticizers; agents for adjusting and fixing the pH; antioxidants; salts; solvents; preserving agents; hair dye precursors; and oxidizing agents.

When present, the amount of these various cosmetic additives and/or of the formulation adjuvants may range from 0.001 to 20% by weight relative to the total weight of the composition.

Those skilled in the art will take care to choose the optional additives and the amount thereof in such a way that they do not harm the advantageous properties of the compositions.

The compositions may also comprise a fatty ester, such as glycerol esters, for example, glyceryl monostearates and glyceryl monocetylate, and fatty acid and fatty alcohol esters such as stearyl esters, myristic esters, cetyl esters, and palmitic esters, esters of stearyl acids, myristic acids, cetylic acids, and palmitic acids.

These esters may be used in an amount ranging from 0.1 to 5%, for example, 0.5 to 3% by weight, relative to the total weight of the composition.

The compositions may be in any form that allows easy application to the hair. Such forms include thickened lotions, aqueous and aqueous-alcoholic gels, creams, and pastes, e.g., hard pastes.

The compositions may be packaged in an aerosol device in the presence of one or more propellants. Propellants include dimethyl ether, C₃₋₅ alkanes, 1,1-difluoroethane, mixtures of dimethyl ether and of C₃₋₅ alkanes, mixtures of 1,1-difluoroethane and of dimethyl ether, and mixtures of 1,1-difluoroethane and of C₃₋₅ alkanes.

The invention is illustrated in greater detail by the examples described below. Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in its respective testing measurements.

### EXAMPLES

### (I) Comparative Examples

In the compositions described below, the percentages are listed by weight of active material.

Conditioner

| | | | 3 | 4 |
|---|---|---|---|---|
| | 1 | 2 | (Composition | (Composition |
| | (Comparative | (Comparative | according to | according to the |
| Ingredients | composition) | composition) | the invention) | invention) |
| Poly(styrene-b- | — | — | 1% | — |
| acrylic acid)[1] | | | | |
| Poly(methyl- | — | — | — | 1% |
| acrylamide-b- | | | | |
| acrylic acid)[2] | | | | |
| Cetearyl alcohol | 3% | 3% | 3% | 3% |
| Cetyl ester | 1% | 1% | 1% | 1% |
| Amodimethicone | 1% | 1% | 1% | 1% |
| DC939 | | | | |
| (Dow Corning) | | | | |
| Behenyltrimonium | — | 1% | — | — |
| chloride | | | | |
| Citric acid/sodium | QS pH 6 | QS pH 6 | QS pH 6 | QS pH 6 |
| hydroxide | | | | |
| Preserving agent | QS | QS | QS | QS |
| Fragrance | QS | QS | QS | QS |
| Water | QSF100% | QSF100% | QSF100% | QSF100% |

[1] Diblock PS (2000 g/mol)-PAA (300 000 g/mol) sold by the company Polymer Source, Inc.

[2] Diblock PAM (5000 g/mol)-PAA (300 000 g/mol) sold by the company Polymer Source, Inc.

12 g of the compositions described above are applied to hair (Caucasian hair 20 cm long having been subjected to at least oxidation dyeing). After two minutes of application, the hair is rinsed with water and the blow dried with a hair dryer. The test is carried out 10 times for each composition. The most relevant cosmetic characteristics of the hair are evaluated by a panel of five experts.

The application of conditioners 1, 2, 3 and 4 as described above is repeated five times. Before each new application of the conditioner, the hair locks are washed with an Elseve Multivitamines shampoo sold by the company L'Oreal. The cosmetic criteria are evaluated by the panel of five experts after the five repeated applications.

The average scores given by the panel of experts are listed in the following tables:

TABLE 1

Composition No.

| | 1 | 2 | 3 | 4 |
|---|---|---|---|---|
| Dentangling on wet hair[a] | 1.9 | 3.8 | 4.1 | 3.6 |
| Dentangling on dry hair[b] | 1.6 | 4.0 | 3.9 | 3.7 |
| Smoothing on dry hair[c] | 1.6 | 3.3 | 3.8 | 3.4 |
| Sheen on dry hair[d] | 2.4 | 3.5 | 3.9 | 3.5 |

The application of the conditioners as described above is repeated ten times. Before each new application of the conditioner, the hair is washed with an Elseve Multivitamines shampoo sold by the company L'Oreal. The cosmetic criteria are again evaluated by the panel of five experts after the ten repeated applications.

The average scores given by the panel of experts are listed in the following table:

TABLE 2

Composition No.

| | 1 | 2 | 3 | 4 |
|---|---|---|---|---|
| Disentangling on wet hair[a] | 2.2 | 3.9 | 4.0 | 3.5 |
| Disentangling on dry hair[b] | 1.8 | 3.8 | 3.8 | 3.7 |
| Smoothing on dry hair[c] | 1.8 | 2.5 | 3.5 | 3.3 |
| Sheen on dry hair[d] | 2.2 | 2.5 | 3.7 | 3.4 |

[a]0: Disentangling not possible
5: Disentangling with a single pass of the comb
[b]0: Disentangling not possible
5: Disentangling with a single pass of the comb
[c]0: uneven feel from root to tip
5: even feel from root to tip
[d]0: dull hair
5: shiny hair

Tables 1 and 2 above show that comparative composition 1 does not confer the expected cosmetic properties of a haircare product.

According to Table 1, after application, the conditioner compositions 3 and 4 give cosmetic properties comparable to comparative composition No. 2 comprising a cationic surfactant.

After ten applications, the test (Table 2) shows that the compositions 3 and 4 give cosmetic properties superior to those of comparative composition (2). For example, levels of sheen and smoothness greater than those obtained with the state of the art compositions are observed for compositions 3 and 4.

The use of the conditioners comprising a water-soluble or water-dispersible block copolymer makes it possible to obtain good cosmetic properties and maintain them at very good levels during multiapplications.

### (II) Examples of Compositions

The following compositions in accordance with the invention are also prepared:

| | | |
|---|---|---|
| Ingredients | Composition 5 | Composition 6 |

| Cetearyl alcohol | 2.5% | A.M. | 2.5% | A.M. |
|---|---|---|---|---|
| Lauryl PEG/PPG-18/18 | 0.23% | A.M. | 0.23% | A.M. |
| Methicone | | | | |
| (Dow Corning 5200) | | | | |
| Cetyl ester | 0.5% | A.M. | 0.5% | A.M. |
| Copolymer (3) | 5% | A.M. | — | |
| Copolymer (4) | — | | 1% | A.M. |
| Amodimethicone | 1.4% | A.M. | 1.4% | A.M. |
| (BELSIL ADM 6057 E - | | | | |
| WACKER) | | | | |

| | | |
|---|---|---|
| Citric acid | — | Qs pH 6 |
| Water | Qsf | Qsf |

Copolymer (3): Diblock copolymer poly(quaternized trimethylammonium ethyl acrylate-b-acrylamide) 11 k-30 k sold by the company Rhodia;

Copolymer (4): Triblock copolymer poly(styrene-b-acrylic acid-b-styrene) 2K-12K-2K (P2971-SAAS) sold by the company Polymer Source, Inc.

What is claimed is:

1. A cosmetic composition comprising, in a cosmetically acceptable medium:
at least one block copolymer comprising at least a first hydrophilic block and at least a second block that is chosen from a second hydrophilic block, wherein the second
hydrophilic block is different than the first hydrophilic block, and a
hydrophobic block, wherein the hydrophilic block(s) represent at least 30% by weight of the at least one block copolymer,
at least one amino silicone, and
at least one nonpolymeric thickener chosen from at least one fatty alcohol and at least one amide,
with the proviso that at least one block copolymer is not chosen from a block copolymer of ethylene oxide and propylene oxide, a block copolymer comprising urethane units, and a block copolymer comprising siloxane units.

2. The composition according to claim 1, wherein the cosmetic composition is a nondetergent cosmetic composition.

3. The composition according to claim 1, wherein the hydrophilic block(s) represent at least 60% by weight of the at least one block copolymer.

4. The composition according to claim 1, wherein the at least one block copolymer is a star copolymer and is chosen from diblock copolymers, triblock copolymers, and multiblock copolymers.

5. The composition according to claim 1, wherein the at least one block copolymer is a linear copolymer and is chosen from diblock copolymers, triblock copolymers, and multiblock copolymers.

6. The composition according to claim 5, wherein the at least one block copolymer is chosen from diblock copolymers and triblock copolymers, wherein the triblock copolymers comprise a hydrophilic central block and two hydrophobic side blocks.

7. The composition according to claim 1, wherein the hydrophilic block(s) are formed from water-soluble monomers chosen from anionic water-soluble monomers, anionic water-dispersible monomers, nonionic water-soluble monomers, nonionic water-dispersible monomers, cationic water-soluble monomers, cationic water-dispersible monomers, and mixtures thereof.

8. The composition according to claim 7, wherein the anionic water-soluble monomers and the anionic water-dispersible monomers are chosen from ethylenically unsaturated carboxylic acids, 2-acrylamido-2-methylpropanesulphonic acid, styrenesulphonic acid, vinylsulphonic acid, and vinylphosphonic acid.

9. The composition according to claim 7, wherein the nonionic water-soluble monomers and the nonionic water-dispersible monomers are chosen from acrylamide, C₁₋₆ N-alkyl acrylamides, C₁₋₃ N,N-dialkyl acrylamides, polyethylene glycol acrylate, polyethylene glycol methacrylate, N-vinylacetamide, N-methyl-N-vinylacetamide, N-vinyl-formamide, N-methyl-N-vinylformamide, N-vinyllactams comprising a cyclic group of 4 to 9 carbon atoms, vinyl alcohol, ethylene oxide, hydroxyethyl acrylate, hydroxypropyl acrylate, hydroxyethyl methacrylate, and hydroxypropyl methacrylate.

10. The composition according to claim 7, wherein the cationic water-soluble monomers and cationic water-dispersible monomers are chosen from dimethyldiallylammonium chloride, methylvinylimidazolium chloride, 2-vinylpyridine, 4-vinylpyridine, 2-methyl-5-vinylpyridine, vinylamine, and monomers of formula:
H₂C═CR₁—CO—X₂
wherein:
R₁ is chosen from hydrogen;
methyl;
a C₁₋₆ linear hydrocarbon-based group bearing at least one substituent chosen from primary amino, secondary amino, tertiary amino, and quaternary amino;
a C₁₋₆ branched hydrocarbon-based group bearing at least one substituent chosen from primary amino, secondary amino, tertiary amino, and quaternary amino;
a group of formula NHR₂; and
a group of formula NR₂R₃;
wherein R₂ and R₃ each are, independently of one another, each chosen from a C₁₋₆ linear hydrocarbon-based group bearing at least one substituent chosen from primary amino, secondary amino, tertiary amino, and quaternary amino; and a C₁₋₆ branched hydrocarbon-based group bearing at least one substituent chosen from primary amino, secondary amino, tertiary amino, and quaternary amino.

11. The composition according to claim 1, wherein the hydrophobic block(s) are formed from water-insoluble monomers chosen from vinylaromatic monomers, dienes, alkylated derivatives of dienes, chloroprene, C₁₋₁₀ alkyl-aralkyl acrylates, C₆₋₁₀ aryl-aralkyl acrylates, C₁₋₁₀ aralkyl acrylates, C₁₋₁₀ alkyl-aralkyl methacrylates, C₆₋₁₀ aryl-aralkyl methacrylates, C₁₋₁₀ aralkyl methacrylates, vinyl acetate, vinyl ethers of formula CH₂═CH—O—R, and allyl ethers of formula CH₂═CH—CH₂—O—R wherein R is chosen from C₁₋₆ alkyl, acrylonitrile, vinyl chloride, vinylidene chloride, caprolactone, ethylene, propylene, fluorinated vinyl monomers, and vinyl monomers comprising a perfluoro chain.

12. The composition according to claim 11, wherein:
the hydrophilic block(s) are formed from water-soluble monomers chosen from anionic water-soluble monomers, water-dispersible monomers, nonionic water-soluble monomers, water-dispersible monomers, cationic water-soluble monomers, water-dispersible monomers, and mixtures thereof, and wherein
the hydrophilic block(s) comprise up to 25 mol % of at least one water-insoluble monomers.

13. The composition according to claim 12, wherein the hydrophilic block(s) contain up to 10 mol % of at least one water-insoluble monomer.

14. The composition according to claim 13, wherein the hydrophilic block(s) contain up to 5 mol % of at least one water-insoluble monomers.

15. The composition according to claim 1, wherein:
the hydrophilic block(s) are formed from water-soluble monomers chosen from anionic water-soluble monomers, water-dispersible monomers, nonionic water-soluble monomers, water-dispersible monomers, cationic water-soluble monomers, water-dispersible monomers, and mixtures thereof, and wherein
the hydrophilic block(s) contain up to 25 mol % of at least one monomer chosen from water-soluble monomers and water-dispersible monomers.

16. The composition according to claim 15, wherein the hydrophilic block(s) contain up to 10 mol % of at least one monomer chosen from water-soluble monomers and water-dispersible monomers.

17. The composition according to claim 16, wherein the hydrophilic block(s) contain up to 5 mol % of at least one monomer chosen from water-soluble monomers and water-dispersible monomers.

18. The composition according to claim 1, wherein the at least one block copolymer is chosen from poly(styrene-b-acrylic acid), poly(methylacrylamide-b-acrylic acid), poly(quaternized trimethylammonium ethyl acrylate-b-acrylamide) diblock copolymer, and poly(styrene-b-acrylic acid-b-styrene) triblock copolymer.

19. The composition according to claim 1, wherein the at least one block copolymer is present in an amount ranging from 0.01 to 20% by weight relative to the composition.

20. The composition according to claim 19, wherein the at least one block copolymer is present in an amount ranging from 0.1 to 5% by weight relative to the composition.

21. The composition according to claim 1, wherein the amino silicone is chosen from:
(a) polysiloxanes of the formula: 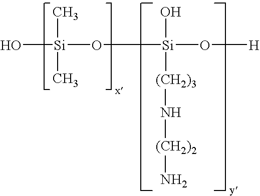
wherein x′ and y′ are each integers chosen such that the weight-average molecular weight ranges from approximately 5000 to 500,000;
(b) amino silicones of the formula:
R′ₐG₃₋ₐ-Si(OSiG₂)ₙ—(OSiG_{b}R′_{2-b})ₘ—O—SiG₃₋ₐ-R′ₐ
wherein:
each G, which may be identical or different, is chosen from hydrogen, phenyl, OH, C₁-C₈ alkyl, and C₁-C₈ alkoxy,
each a, which may be identical or different, is a number chosen from 0, 1, 2, and 3,
b is chosen from 0 and 1,
m and n are numbers chosen such that the sum (n+m) ranges from 1 to 2000,
each R′, which may be identical or different, is a monovalent radical of the formula —C_{q}H_{2q}L wherein q is a number chosen from 2, 3, 4, 5, 6, 7, and 8 and L is an optionally quaternized amino group chosen from:
—NR″-Q-N(R″)₂,
—N(R″)₂,
—N⁺(R″)₃A⁻,
—N⁺H(R″)₂A⁻,
—N⁺H₂(R″)A⁻,
—N(R″)-Q-N⁺R″H₂A⁻,
—NR″-Q-N⁺(R″)₂H A⁻, and
—NR″-Q-N⁺(R″)₃A⁻,
wherein R″ is chosen from hydrogen, phenyl, benzyl, and a monovalent saturated hydrocarbon-based radical,
Q is a linear or branched group of formula CᵣH₂ᵣ wherein r is an integer chosen from 2, 3, 4, 5, and 6; and
A⁻ is a halide ion;
(c) amino silicones of the formula: 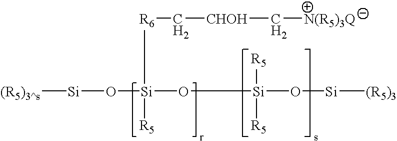
wherein:
R₅ is a monovalent hydrocarbon-based radical having from 1 to 18 carbon atoms,
R₆ is a divalent hydrocarbon-based radical connected to the Si via a silicon-carbon bond;
Q⁻ is chosen from an anion and carboxylate (aren't carboxylate anions? Be careful of redundancies.};
r is a mean statistical value ranging from 2 to 20; and
s is a mean statistical value ranging from 20 to 200;
(d) quaternary ammonium silicones of the formula: 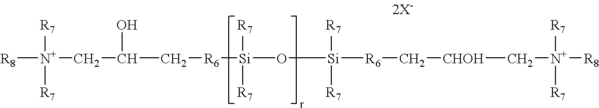
wherein:
each R₇, which may be identical or different, is a monovalent hydrocarbon-based radical having from 1 to 18 carbon atoms;
each R₆, which may be identical or different, is a divalent hydrocarbon-based radical connected to silicon via a silicon-carbon bond;
each R₈, which may be identical or different, is chosen from hydrogen and a monovalent hydrocarbon-based radical having from 1 to 18 carbon atoms;
X⁻ is chosen from an anion and a carboxylate; and
r is a mean statistical value ranging from 2 to 200;
(e) amino silicones of the formula: 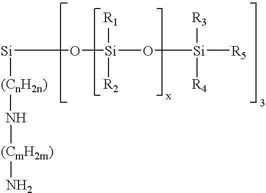
wherein:
R₁, R₂, R₃ and R₄, which may be identical or different, are each chosen from C₁-C₄ alkyl and phenyl,
R₅ is chosen from C₁-C₄ alkyl and hydroxyl,
n is an integer chosen from 1, 2, 3, 4, and 5,
m is an integer chosen from 1, 2, 3, 4, and 5, and
x is a number chosen such that the amine number ranges from 0.01 and 1 meq/g; and
(f) polyoxyalkylenated amino silicones of the formula
(XY)ᵢ
wherein:
X is a polysiloxane block, and
Y is a polyoxyalkylenated block comprising at least one amine group.

22. The composition according to claim 21, wherein the amino silicone is chosen from polyoxyalkylenated amino silicones of the formula:
(XY)ᵢ
wherein:
Y is a polyoxyalkylenated block comprising at least one amine group. comprising repeat units of the formula:
[SiMe₂—O—(SiMe₂O)ₓSiMe₂—R—N(H)—R′—O—(C₂H₄O)ₐ—(C₃H₆O)_{b}—R′—N(H)—R-]
wherein:
a is an integer greater than or equal to 1;
b is an integer ranging from 0 to 200;
x is an integer ranging from 1 to 10,000;
each R, which may be identical or different, is a divalent organic group linked to the adjacent silicon atom via a carbon-silicon bond and to a nitrogen atom;
each R′, which may be identical or different, is a divalent organic group linked to the adjacent oxygen atom via a carbon-oxygen bond and to a nitrogen atom.

23. The composition according to claim 1, wherein the at least one amino silicone is present in an amount ranging from 0.01 to 20% by weight.

24. The composition according to claim 23, wherein the at least one amino silicone is present in an amount ranging from 0.1 to 15% by weight.

25. The composition according to claim 1, wherein the at least one fatty alcohol is chosen from stearyl alcohol, myristic alcohol, cetyl alcohol, and mixtures thereof.

26. The composition according to claim 1, wherein the at least one amide comprises a C₁₂-C₂₂ fatty chain.

27. The composition according to claim 26, wherein the at least one amide comprises a C₁₆-C₁₈ fatty chain.

28. The composition according to claim 1, wherein the at least one nonpolymeric thickener is used in an amount ranging from 0.5 to 10% by weight, relative to the total weight of the composition.

29. The composition according to claim 28, wherein the at least one nonpolymeric thickener is used in an amount ranging from 2 to 6% by weight, relative to the total weight of the composition.

30. The composition according to claim 1, further comprising at least one ingredient chosen from anionic surfactants, cationic surfactants, amphoteric surfactants, nonionic surfactants, anionic polymers, cationic polymers, amphoteric polymers, and nonionic polymers, pearlescent agents, opacifiers, pigments, dyes, fragrances, plant oils, animal oils, synthetic oils, silicone gums, silicone resins, waxes, organic and mineral microparticles, organic and mineral nanoparticles, organic UV-screening agents, mineral UV-screening agents, free-radical scavengers, vitamins, peptides, proteins, amino acids, antidandruff agents, antifungal agents, agents for activating hair regrowth, plasticizers, agents for adjusting the pH, agents for fixing the pH, antioxidants, salts, solvents, preserving agents, hair dye precursors, and oxidizing agents.

31. The composition according to claim 1, in a form chosen from a thickened lotion, gel, cream, and paste.

32. A method of disentangling, smoothing, and imparting sheen to hair comprising applying the composition of claim 1 to hair.

33. A method of imparting good cosmetic properties to hair that are maintained after multiple applications comprising applying the composition of claim 1 to the hair.
